(19) United States
(12) Patent Application Publication
(10) Pub. No.: US 2026/0253338 A1
(43) Pub. Date: Aug. 27, 2026

(54) EFFICIENT RIB PLANE VISUALIZATION VIA AUTO AUGMENTATION

(71) Applicant: KONINKLIJKE PHILIPS N.V., EINDHOVEN (NL)

(72) Inventors: TOBIAS KLINDER, UELZEN (DE); CRISTIAN LORENZ, HAMBURG (DE); CHRISTIAN BUERGER, HAMBURG (DE); TANJA LOSSAU, HAMBURG (DE); ALENA-KATHRIN GOLLA, QUICKBORN (DE); ZHANGPENG HAO, SHENYANG (CN)

(21) Appl. No.: 18/994,102

(22) PCT Filed: Jul. 13, 2023
(86) PCT No.: PCT/EP2023/069411

§ 371 Date: Jan. 14, 2025

Related U.S. Application Data
(60) Provisional application No. 63/389,956, filed on Jul. 18, 2022.

(30) Foreign Application Priority Data
Jul. 22, 2022 (WO) PCT/CN2022/107254

(51) Int. Cl. G06T 19/00; G06T 7/12; G16H 30/40

(52) U.S. Cl. CPC G06T 19/00; G06T 7/12; G16H 30/40; G06T 2210/41

(57) ABSTRACT

A rib plane visualization based on axial views is augmented. Data representative of a three-dimensional diagnostic image is received. The ribs are segmented, and rib centerlines are detected and labeled. A fit plane corresponding to the rib centerline is calculated separately for each rib, and out-of-plane rib parts are determined. A visualization of the ribs is generated individually or as rib pairs. An augmented visualization is generated for out-of-plane rib parts and may be performed via a fusion scheme or a curved surface scheme. The fusion scheme includes projecting out-of-plane rib parts onto the visualization as fused with the in-plane rib parts. The curved surface scheme includes fitting a polynomial surface, such that all points in a point set will be close to the surface. With this approach, individual rib (pairs) can easily be inspected in single axial views.

### FIELD OF THE INVENTION

The invention relates to the field of medical imaging and, in particular, a method and apparatus for rib plane visualization via auto augmentation.

### BACKGROUND OF THE INVENTION

Reading imaging scans and, more specifically, trauma or Emergency Department scans is a time-critical task that needs to be done with high attention to avoid overlooking critical findings. Often, in these settings, imaging is based on whole-body scans which results in large amounts of imaging data being generated, and the large amounts of imaging data generated must be thoroughly inspected. The ribs are especially critical structures to be assessed, e.g. with respect to trauma. These are repetitive structures which take a significant amount of time to examine (commonly 12 rib pairs and 24 vertebrae).

Computed Tomography (CT) scans have become the modality of choice to assess the overall condition of a patient for applications, such as in a trauma or emergency setting. Medical personal, such as a radiologist, often rely on medial image data in the form of a CT scan for diagnostic purposes, for example the detection of rib fractures. During reading, each of the 24 ribs needs to be followed individually while scrolling through the image slices.

Looking at thoracic or whole-body three-dimensional (3D) CT scans slice by slice is often a time-consuming process, especially when the target anatomical structure spans multiple slices (e.g., 24 individual ribs to trace). As a result, rib abnormalities are likely overlooked.

Assessment of the ribs require significant reading time, as the ribs are commonly traced through the volume of the image data one by one. In addition, pathologies such as fractures (especially buckle) can be very subtle and easily overlooked.

Several approaches to simplify the visualization and evaluation of a patient's anatomy and critical structures have been proposed, especially those targeting visualization of the rib cages. While providing some benefit, current approaches come with distinct fundamental limitations.

One well-known visualization scheme is the “filet view” or “fishbone view.” This view is based on segmenting the ribs (e.g., using a deep convolutional neural network) followed by a centerline extractor which subsequently labels rib pairs in the field of view. Each rib is sampled along its trace allowing a visualization of each and every rib in a normalized and straightened manner (curved planar reformat). This view allows medical professionals, such as a radiologist, to accurately inspect the rib centerlines in a normalized view where all the ribs are straightened and placed in a unique position on the inspection canvas (reformatted view).

There are, however, several shortcomings with this type of view. One disadvantage with this type of view is that the nature of processing each rib independently leads to discontinuities between ribs, which may lead to imaging artifacts from adjacent ribs appearing in rib shapes.

Another visualization scheme is the visceral cavity view. In this view, a segmentation algorithm (e.g., using a model-based approach) is applied to segment the inside of the rib cage in terms of a deformation of a cylindric manifold. Once segmentation is done, the manifold may be unwrapped and a maximum intensity projection (MIP) close to the proximity of the surface may be computed. This view allows the user to inspect the rib cage as a whole in terms of a continuous visualization on the inspection canvas.

One of the deficiencies with this type of view is that the relative rib lengths are not maintained. In other words, the nature of unwrapping a cylinder manifold does not allow visualization of the correct rib lengths. For example, the first rib appears too long in relation to the other ribs. In addition, the nature of the MIP visualization may not allow detection of subtle fractures. For instance, the MIP visualization may make small rib fractures invisible and not detectable in the generated view. Another deficiency is that this view adds significant unrealistic distortions (wavy ribs) which limit clinical confidence in the generated visceral cavity view.

Thus, the need exists for an efficient rib plane visualization that overcomes the disadvantages associated with these views.

### SUMMARY OF THE INVENTION

The object of the present invention provides techniques for an automatic augmentation view that overcomes deficiencies in existing visualization schemes. The techniques may be applied to a number of imaging systems including CT, CT-arm, Single Photon Emission Computed Tomography CT (SPECT-CT), Magnetic Resonance CT (MR-CT), Positron Emission Tomography CT (PET-CT), and Magnetic Resonance Imaging (MRI) systems.

According to a first aspect of the invention a method of augmenting a rib plane visualization is provided. The method includes receiving data representative of a three-dimensional diagnostic image, the three-dimensional diagnostic image including one or more ribs of a subject; segmenting the one or more ribs according to the received data representative of the three dimensional diagnostic image; detecting and labeling rib centerlines from the rib segmentation; calculating separately for each rib a fit plane corresponding to the rib centerline; determining out of plane rib parts for each rib; generating a visualization of the ribs individually or as rib pairs; and generating an augmented visualization for out of plane rib parts.

In a second aspect of the invention, an imaging apparatus is provided. The imaging apparatus comprising a memory configured to store computer executable instructions and at least one processor configured to execute the computer executable instructions to cause the imaging apparatus to: receive data representative of a three-dimensional diagnostic image, the three-dimensional diagnostic image including one or more ribs of a subject; segment the one or more ribs according to the received data representative of the three dimensional diagnostic image; detect and label rib centerlines from the rib segmentation; calculate separately for each rib a fit plane corresponding to the rib centerline; determine out of plane rib parts for each rib; generate a visualization of the ribs individually or as rib pars; and generate an augmented visualization for out of plane rib parts.

In a third aspect of the invention, a non-transitory computer-readable medium having stored thereon instructions for causing processing circuitry to execute a process is provided. The process comprising receiving data representative of a three-dimensional diagnostic image, the three-dimensional diagnostic image including one or more ribs of a subject; segmenting the one or more ribs according to the received data representative of the three dimensional diagnostic image; detecting and labeling rib centerlines from the rib segmentation; calculating separately for each rib a fit plane corresponding to the rib centerline; determining out of plane rib parts for each rib; generating a visualization of the ribs individually or as rib pars; and generating an augmented visualization for out of plane rib parts.

In a preferred embodiment, generating an augmented visualization for out of plane rib parts comprises computing a distance to fit plane for each rib centerline point, and wherein rib parts outside the fit plane are projected onto the visualization of the rib as being fused with the in plane rib parts. The out of plane rib parts may be highlighted to identify rib parts that are not part of an original visualization of the rib. An aspect of the invention provides for switching between an original visualization of the rib and the augmented visualization of the rib, as well as highlighting an influence radius in the augmented visualization of the rib.

In a preferred embodiment, generating an augmented visualization for out of plane rib parts comprises establishing a point set that includes all centerline points of a rib and cross points between the fit plane and a data boundary and fitting a polynomial surface, such that all points in the point set will be close to the surface. In an aspect of the invention, a render direction is set to be the same as the fit plane, the surface is rendered, and the rendering result is projected to the fit plane. The render mode may be selected from the group consisting of a Multiplanar Reformation (MPR), a Maximum Intensity Projection (MIP), a Minimum Intensity Projection (MinIP), an Average Intensity Projection (AIP), and a Volume Rendering (VR).

In an aspect of the invention, segmenting the ribs and detecting and labeling rib centerlines is performed via machine learning or deep learning techniques.

### BRIEF DESCRIPTION OF THE DRAWINGS

In the following drawings:

FIG. 1 is a diagram of an exemplary imaging system;

FIG. 2 illustrates a view of a rib cage according to the “filet view” or “fishbone view” and visceral cavity view.

FIG. 3 illustrates rib centerline points in a single plane;

FIG. 4 illustrates a visualization of ribs based on a tilted axis view corresponding to a fitted plane;

FIG. 5 illustrated parts of the ribs locally out of plane that are automatically detected;

FIG. 6 illustrates an augmented view;

FIG. 7 illustrates an MPR view and an augmented view according to some embodiments;

FIG. 8 illustrates a surface fit;

FIG. 9 illustrates an augmented view according to some embodiments;

FIG. 10 is a flowchart representing according to some embodiments;

FIG. 11 is a flowchart representing a method of augmenting a rib plane visualization via a fusion scheme according to some embodiments; and

FIG. 12 is a flowchart representing a method of augmenting a rib plane visualization via a curved surface scheme according to some embodiments.

### DETAILED DESCRIPTION OF EMBODIMENTS

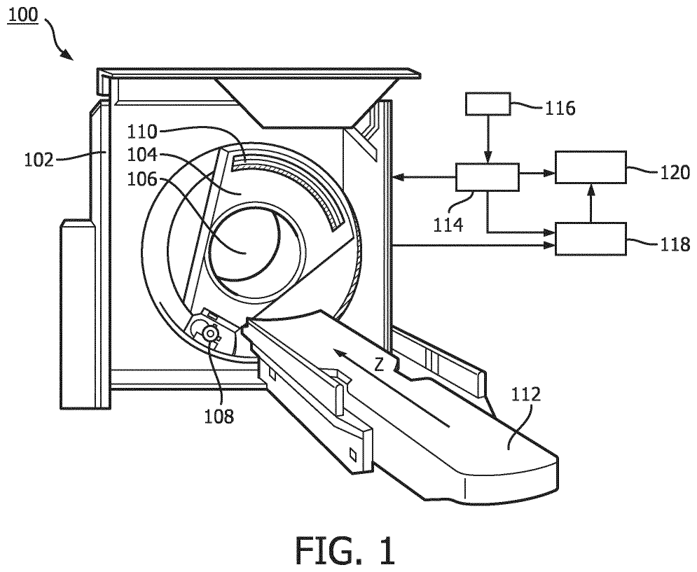

FIG. 1 illustrates an imaging system 100, such as a computed tomography (CT) imaging system. The imaging system 100 includes a generally stationary gantry 102 and a rotating gantry 104. The rotating gantry 104 is rotatably supported by the stationary gantry 102 and rotates in relation to the stationary gantry 102 around an examination region 106 about a longitudinal or z-axis.

A patient support 112, such as a couch, supports an object or examination subject, such as a human patient, in an examination region 106. The support 112 is configured to move the object or examination subject for loading, scanning, and unloading.

A radiation source 108, such as an x-ray tube, is rotatably supported by the rotating gantry 104. The radiation source 108 rotates with the rotating gantry 104 and emits radiation that traverses the examination region 106.

A radiation sensitive detector array 110 subtends an angular arc opposite the radiation source 108 across the examination region 106. The detector array 110 includes one or more rows of detectors that extend along the z-axis direction, detect radiation traversing the examination region 106, and generates projection data indicative thereof.

A general-purpose computing system or computer serves as an operator console 114 and includes an input device(s) 116, such as a mouse, a keyboard, and/or the like and an output device(s) 120, such as a display monitor, a filmer or the like. The console 114 allows an operator to control the operation of the system 100. This includes control of an imaging processing device 118. The imaging processing device 118 may receive data representative of a three-dimensional (3D) diagnostic image generated by the imaging system 100, calculate, separately for each rib, a fit plane corresponding to the rib centerline, generate a visualization of the ribs, and generate an augmented visualization when rib parts lie outside a determined fit plane. A user may toggle between the original visualization of a given rib and an augmented visualization of the rib.

It is to be appreciated that the processing of imaging processing device 118 can be implemented through a microprocessor(s), which executes a computer readable instruction(s) encoded or embedded on computer readable storage medium, such as physical memory and other non-transitory medium. Additionally or alternatively, the microprocessor(s) can execute a computer readable instruction(s) carried by a carrier wave, a signal and other transitory medium.

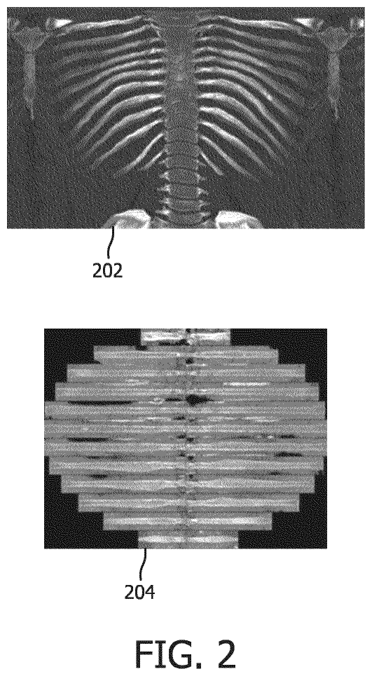

FIG. 2 illustrates various views of a rib cage according to a visceral cavity view 202 and a “filet view” or “fishbone view” 204. It can be seen that these views allow a medical professional, such as a radiologist, to accurately inspect the rib centerlines in an unfolded view (reformatted view) where all the ribs are straightened and placed into a unique position on the inspection canvas. These views allow an efficient assessment of the overall rib cage, but they introduce a new view that radiologists need to become familiar with and can show significant distortions and even hide pathologies, such as fractures.

The novel visualization scheme presented is based on axial view plane adjustments. The axial plane is the view that most radiologist use to primarily interpret related imaging studies. Usually most parts of the ribs lie within a single plane. To make use of this property, a reformat is shown to the user which corresponds to an optimal view plane. A tilted axial view can be easily inspected by a radiologist. Parts of the rib, however, might not lie within the plane and therefore may be overlooked.

Thus, the novel auto augmentation scheme automatically detects out-of-plane parts to yield augmented views that allow inspection of the out-of-plane parts. Two different implementations of augmented views are provided, and affected parts may be highlighted to allow the user to identify the rib parts that are not part of the original visualization of the rib.

One augmented view is provided by performing fusion of parts that do not lie within the plane. Another augmented is provided by generating a curved surface that is close to the tilted axial plane and rib centerline points.

For each augmented view, segmentation of the ribs is performed, rib centerlines are extracted, and a fit plane corresponding to the rib centerline is calculated separately for each rib a fit plane.

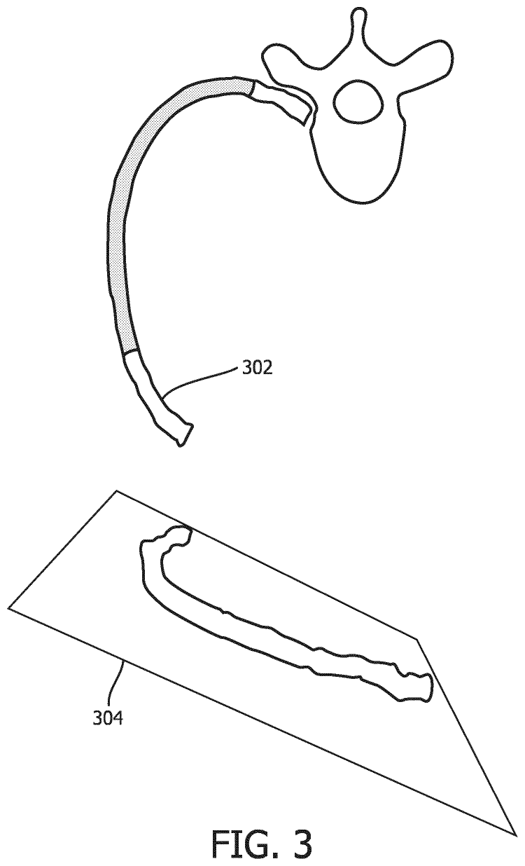

FIG. 3 illustrates centerline points of a rib in a single plane. From segmentation, several different representations may be derived. A rib centerline visualization is a common representation derived from the segmentation. The segmentations and/or derived representations can be labelled according to anatomical label (i.e. L1-12, R1-12). For segmentation, labelling and centerline extraction several well-established methods exist and have successfully proven sufficient robustness. One example of such approach is applying a fully convolutional neural network (FCNN) to generate a probability map for detecting the first rib pair, the twelfth rib pair, and the collection of all intermediate ribs. In a second stage, a centerline extraction algorithm is applied to this multi-label probability map. Finally, the distinct detection of a first and a twelfth rib separately allows for deriving individual rib labels by sorting and counting the detected centerlines. The rib centerlines may obtained using interpolation schemes such as splines.

Typically, most of the centerline points of a rib will lie approximately within a single plane. The plane may be determined via covariance analysis of the coordinates of the center points where the eigenvector corresponding to the smallest eigenvalue of the covariance matrix corresponds to the plane normal vector. As the rib, especially at the vertebra connection and close to the sternum, often show significant bending, a weighted covariance matrix focusing on the central part of the ribs is beneficial to ensure that the calculated plane passes through the majority of points.

Different weighing of centerline points for the plane fit calculation are illustrated at 302. Lighter parts have lower weights to ensure focus on the major part of the ribs. A plane fit through the rib, shown as a mesh, is illustrated at 304.

For the fusion scheme, using the calculated plane fit, out-of-plane parts can be automatically determined by calculating for each centerline the distance d to the found plane. All points larger than a specified threshold T will be considered out of plane. Based on the determined distances, two major scenarios may be defined.

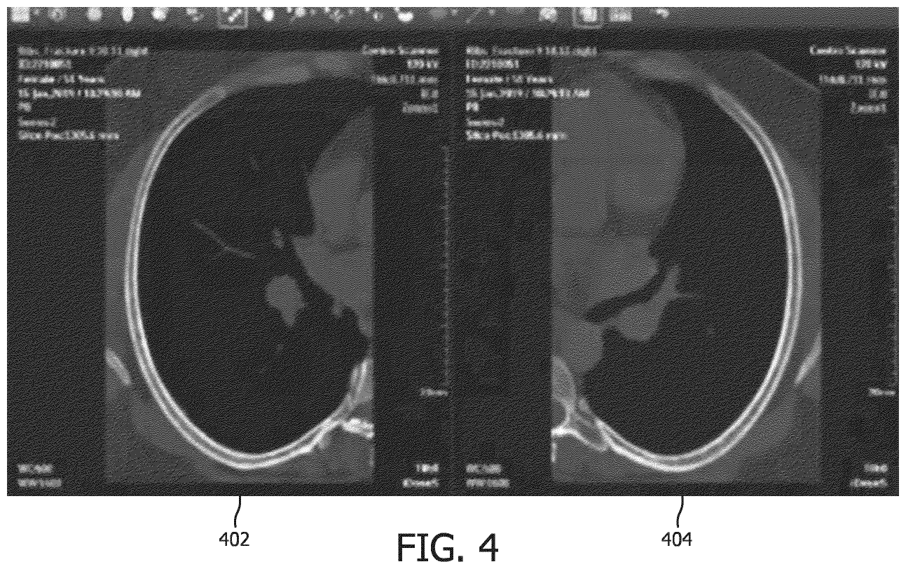

FIG. 4 illustrates the first scenario. In the first scenario, all points lie withing or close enough to the plane. By visualizing a titled axial view that corresponds to the determined plane fit, the ribs can be nicely assessed. A visualization of a rib pair 402 and 404 based on a tilted axial view corresponding to the fitted plane is shown in FIG. 4.

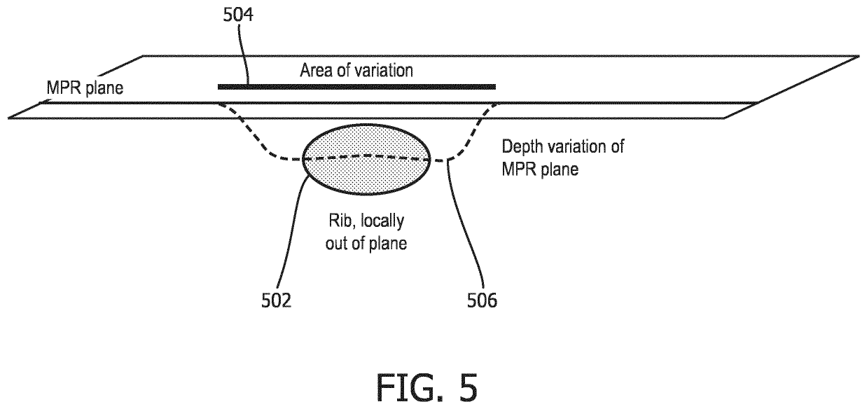

FIG. 5 illustrates the second scenario. The second scenario is the case where certain points lie outside the plane. In such a case, certain parts of the ribs cannot be assessed with the titled axial derived from the plane fit. In these cases the user starts navigation to display the out-of-the-plane part. Parts originally within the plane, however, will disappear. This adds complexity and reading time of the image slices. Thus, in such a scenario where not all parts are within the plane an “augmented view” is implemented. Parts within the plane are displayed as before, but parts outside of the plane are projected into the plane. More precisely, as ribs are bright structures, a maximum intensity projection can be performed locally. By smoothly transitioning this projection, sharp edges are prevented in the image.

Parts of the ribs outside of the Multiplanar Reformation (MPR) are automatically determined at 502. A larger area of variation along the rib is determined at 504, and the depth variation of the MPR plane 506 is varied smoothly to guarantee a smooth resulting image. Fusion is limited to only the area where the rib is locally out of plane. Thus, most of the image is not affected. The fused parts may be highlighted, for example via a color overlay or colored circle, to identify to the user the region or regions that are not part of the original image.

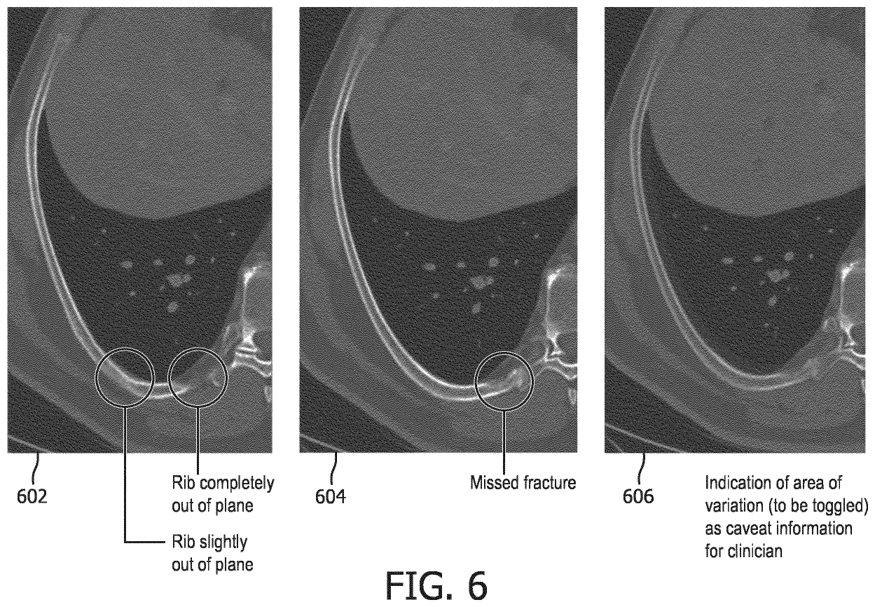

FIG. 6 illustrates out-of-plane rib parts and an augmented view of the out of plane parts. A MPR in which rib parts slightly out-of-plane and completely out-of-plane is shown at 602. A missed fracture is shown in an augmented view at 604. The highlighting may be extended to the whole region that may potentially be considered to show the influence radius. An indication of the area of variation is shown at 606. This may be provided as caveat information to a clinician or radiologist, and the highlighting may be toggled on and off by the clinician.

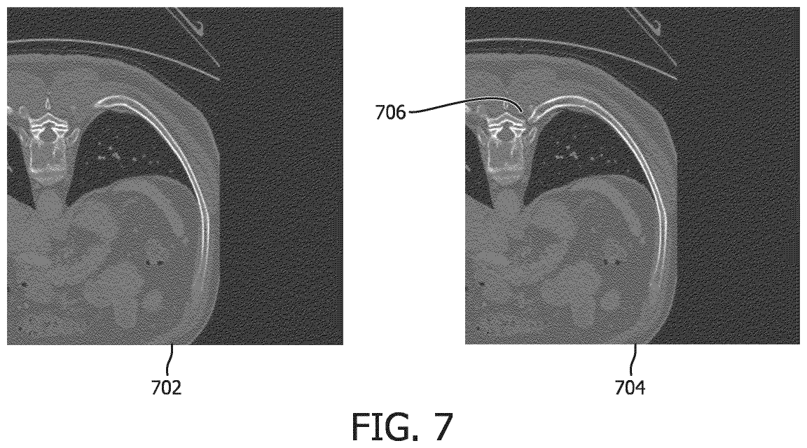

Another example of an MPR view and an augmented view is illustrated in FIG. 7. An MPR view is shown at 702, and an augmented view is shown at 704. The area at 706 of augmented view 704 illustrates an out-of-plane part of the rib missing in the MPR view 702.

For the curved surface scheme, with an understanding that there are countless surfaces that could cross centerline points, given a family of surface equations the closest surface may not be close to the fit plane (tilted axial plane). To ensure that the surface is close to that plane, it is necessary to extend the point set with points that are located on the plane. This limits any potential irregularity of the surface so that the image content will be more natural.

A polynomial equation is a way to construct and solve the curved surface, and the curved part could be easily controlled through the high-order parts. The accuracy of the surface may be improved by adding high-order parts and limit the bending by adding a penalty to high-order part. The order of polynomial equation may be determined by limiting the average distance between points set and the calculated surface.

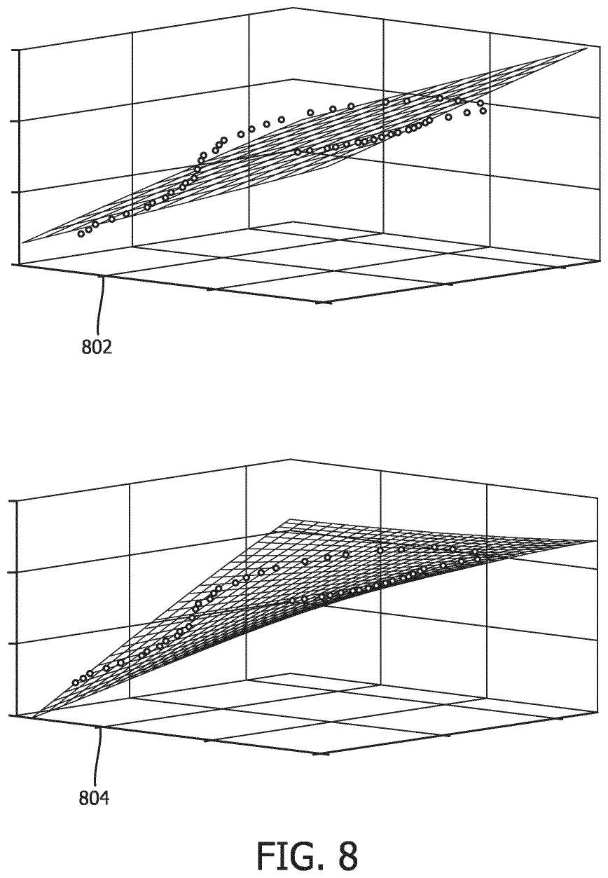

FIG. 8 illustrates a surface fit. A fit plane in which parts of the points are locally out of the plane is shown at 802. An augmented view via surface fitting is shown at 804. The fitted polynomial surface is close to the original fit plane, and the distance between points and the surface is reduced.

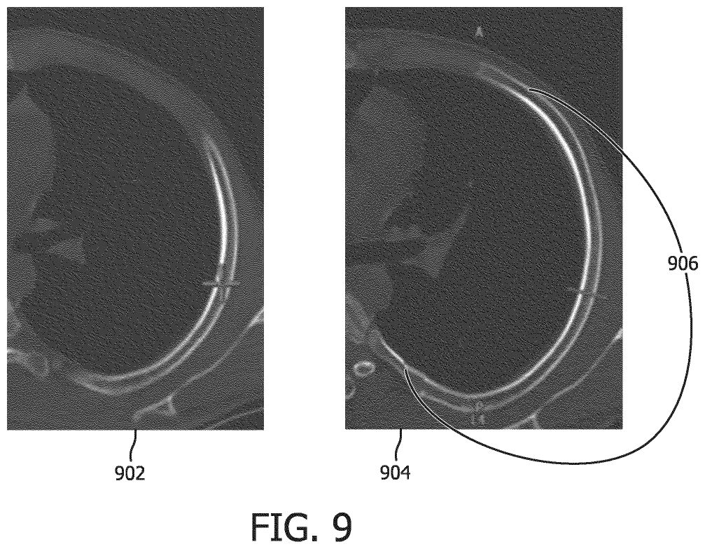

FIG. 9 illustrates an augmented view with a surface fit. At 902 an MPR in which parts of the ribs are locally out of plane is shown. An augmented view is shown at 904. The augmented view shows out-of-plane parts in areas 906 that are missing in the MPR 902.

Several different render modes, for example Maximum Intensity Projection (MIP), a Minimum Intensity Projection (MinIP), an Average Intensity Projection (AIP), and a Volume Rendering (VR), may be applied verifying that the full image shares the same thickness.

For both the fusion approach and the curved surface approach plane fits will be calculated for each rib separately in order to efficiently navigate through the entire rib cage. Left and right ribs may be displayed side-by-side as illustrated in FIG. 4. Overview images may be shown for all 12 rib pairs in one stacked overview. Interaction may be enabled to click from one rib pair to the next. For each rib, individual navigation is possible (preferably, axis-parallel and limited to the rib).

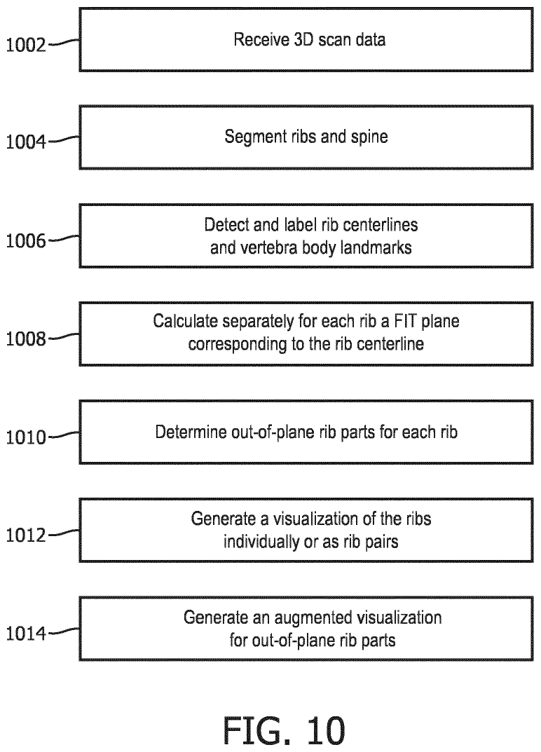

FIG. 10 is a flowchart representing a method of augmenting a rib plane visualization according to some embodiments. At 1002 data representative of a three dimensional (3D) diagnostic image including ribs of a subject, for example a human patient, is received.

At 1004 the ribs are segmented according to the received data representative of the 3D diagnostic image. Segmenting the ribs may be performed manually, semi-automatically, or by applying an automated machine learning approach such as a fully convolutional neural network (FCNN) to generate a probability map for detecting the first rib pair, the twelfth rib pair, and the collection of all intermediate ribs. Next, a centerline extraction algorithm is applied to this multi-label probability map. The rib centerlines may obtained using cubic spline interpolation. Finally, the distinct detection of a first and a twelfth rib separately allows for deriving individual rib labels by simple sorting and counting the detected centerlines.

Rib centerlines are detected and labeled from the from the rib segmentation at 1006. The segmenting of the ribs may be performed via machine learning or deep learning techniques.

At 1008 a fit plane corresponding to the rib centerline is calculated separately for each rib. Out-of-plane rib parts are determined for each rib at 1010. At 1012 a visualization of the ribs is generated. The visualization at 1012 may be generated for each rib individually or as rib pairs. An augmented visualization for out-of-plane rib parts is generated at 1014.

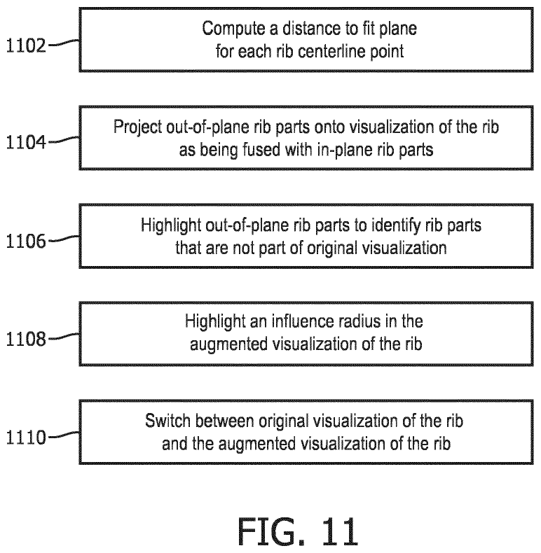

FIG. 11 is a flowchart representing a method of augmenting a rib plane visualization via a fusion scheme according to some embodiments. At 1102 a distance to fit plane for each rib centerline point is computed. That is, for each centerline the distance d to the found plane is computed. In a case where all points lie within or close to the plane, the rib(s) may be assessed visualizing a tilted axial view that corresponds to the determined plane. This is shown in FIG. 4 at 402.

For the case where certain points lie outside of the plane, the associated out-of-plane parts of the rib cannot be assessed with the tilted axial derived from the fit plane. Using the calculated fit plane, out-of-plane rib parts may be automatically determined. The automatically determined out-of-plane rib parts are projected onto the visualization of the rib and viewed as being fused with the in-plane parts of the rib at 1104 as shown in FIG. 4 at 402. The fusion is limited to the area where the rib is locally out-of-plane so that most of the image region that includes the area where the rib is in-plane is not affected.

At 1106 out-of-plane rib parts may be highlighted in the augmented view to identify rib parts that are not part of the original visualization. At 1108 an influence radius may be highlighted in the augmented visualization of the rib. The user may switch between the original visualization of the rib and the augmented visualization of the rib at 1110.

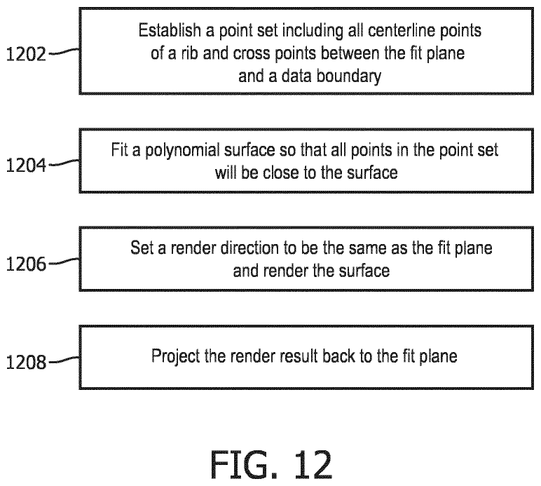

FIG. 12 is a flowchart representing a method of augmenting a rib plane visualization via a curved surface scheme according to some embodiments. At 1202 a point set including all centerline points of a rib and cross points between the fit plane and a data boundary is established.

Considering that there are countless surfaces that could cross centerline points, given a family of surface equations, the closest surface may not be close to the fit plane. In order to ensure that the surface is close to the plane, a point set may be extended with points that are located on the plane. A polynomial equation is an effective way to construct the curved surface. At 1204 a polynomial surface is fit such that all points in the point set will be close to the surface.

The order of the polynomial equation is determined by limiting the average distance between the points set and the calculated surface. The accuracy of the surface may be improved by adding high-order parts. At 1206 a render direction is set to be the same as the fit plane surface. FIG. 9 shows an MPR rendering and augmented view according to a curved surface scheme. Different render modes including MIP, MinIP, AIP, and VR, may also be applied. At 1208 the rendered result is projected to the fit plane.

While the invention has been illustrated and described in detail in the drawings and foregoing description, such illustrations and description are to be considered illustrative or exemplary and not restrictive; the invention is not limited to the disclosed embodiments.

Other variations to the disclosed embodiments can be understood and effected by those skilled in the art in practicing the claimed invention, from a study of the drawings, the disclosure, and the appended claims.

In the claims, the word “comprising” does not exclude other elements or steps, and the indefinite article “a” or “an” does not exclude a plurality.

A single processor, device or other unit may fulfill the functions of several items recited in the claims. The mere fact that certain measures are recited in mutually different dependent claims does not indicate that a combination of these measures cannot be used to advantage.

Operations like acquiring, determining, obtaining, outputting, providing, store or storing, calculating, simulating, receiving, warning, and stopping can be implemented as program code means of a computer program and/or as dedicated hardware.

A computer program may be stored and/or distributed on a suitable medium, such as an optical storage medium or a solid-state medium, supplied together with or as part of other hardware, but may also be distributed in other forms, such as via the Internet or other wired or wireless telecommunication systems.

1. A method of augmenting a rib plane visualization, the method comprising:
receiving data representative of a three-dimensional diagnostic image, the three-dimensional diagnostic image including one or more ribs of a subject;
segmenting the one or more ribs according to the received data representative of the three dimensional diagnostic image;
detecting and labeling rib centerlines from the rib segmentation;
calculating separately for each rib a fit plane corresponding to the rib centerline;
determining out of plane rib parts for each rib;
generating a visualization of the ribs individually or as rib pars; and
generating an augmented visualization for out of plane rib parts.

2. The method according to claim 1, wherein generating an augmented visualization for out of plane rib parts comprises computing a distance to fit plane for each rib centerline point, and wherein rib parts outside the fit plane are projected onto the visualization of the rib as being fused with the in plane rib parts.

3. The method according to claim 2, wherein the out of plane rib parts are highlighted to identify rib parts that are not part of an original visualization of the rib.

4. The method according to claim 2, further comprising switching between an original visualization of the rib and the augmented visualization of the rib.

5. The method according to claim 2, further comprising highlighting an influence radius in the augmented visualization of the rib.

6. The method according to claim 1, wherein generating an augmented visualization for out of plane rib parts comprises establishing a point set that includes all centerline points of a rib and cross points between the fit plane and a data boundary and fitting a polynomial surface such that all points in the point set will be close to the surface.

7. The method according to claim 6, further comprising setting a render direction to be the same as the fit plane, rendering the surface, and projecting the rendering result to the fit plane.

8. The method according to claim 7, wherein a render mode is selected from the group consisting of a Multiplanar Reformation (MPR), a Maximum Intensity Projection (MIP), a Minimum Intensity Projection (MinIP), an Average Intensity Projection (AIP), and a Volume Rendering (VR).

9. The method according to claim 1, wherein segmenting the ribs and detecting and labeling rib centerlines is performed via machine learning or deep learning techniques.

10. An imagining apparatus comprising:
a memory configured to store computer executable instructions; and
at least one processor configured to execute the computer executable instructions to cause the imaging apparatus to:
receive data representative of a three-dimensional diagnostic image, the three-dimensional diagnostic image including one or more ribs of a subject;
segment the one or more ribs according to the received data representative of the three dimensional diagnostic image;
detect and label rib centerlines from the rib segmentation;
calculate separately for each rib a fit plane corresponding to the rib centerline;
determine out of plane rib parts for each rib;
generate a visualization of the ribs individually or as rib pars; and
generate an augmented visualization for out of plane rib parts.

11. The imagining apparatus according to claim 10, wherein the processor is configured to generate an augmented visualization for out of plane rib parts by computing a distance to fit plane for each rib centerline point, and project rib parts outside the fit plane onto the visualization of the rib as appearing fused with the in plane rib parts.

12. The imagining apparatus according to claim 11, wherein the processor is configured to highlight the out of plane rib parts to identify rib parts that are not part of an original visualization of the rib.

13. The imagining apparatus according to claim 11, wherein the processor is further configured to switch between an original visualization of the rib or rib pair and the augmented visualization of the rib or rib pair based on a user input.

14. The imagining apparatus according to claim 11, wherein the processor is further configured to highlight an influence radius in the augmented visualization of the rib or rib pair based on a user input.

15. The imagining apparatus according to claim 10, wherein the processor is configured to generate an augmented visualization for out of plane rib parts by establishing a point set that includes all centerline points of a rib and cross points between the fit plane and a data boundary and fitting a polynomial surface such that all points in the point set will be close to the surface.

16. The imagining apparatus according to claim 15, wherein the processor is further configured to set a render direction to be the same as the fit plane, render the surface, and project the render result to the fit plane.

17. The imagining apparatus according to claim 16, wherein the processor is configured to select a render mode from the group consisting of a Multiplanar Reformation (MPR), a Maximum Intensity Projection (MIP), a Minimum Intensity Projection (MinIP), an Average Intensity Projection (AIP), and a Volume Rendering (VR).

18. The imagining apparatus according to claim 10, wherein segmenting the ribs and detecting and labeling rib centerlines is performed via machine learning or deep learning techniques.

19. A non-transitory computer-readable medium having stored thereon instructions for causing processing circuitry to execute a process, the process comprising:
receiving data representative of a three-dimensional diagnostic image, the three-dimensional diagnostic image including one or more ribs of a subject;
segmenting the one or more ribs according to the received data representative of the three dimensional diagnostic image;
detecting and labeling rib centerlines from the rib segmentation;
calculating separately for each rib a fit plane corresponding to the rib centerline;
determining out of plane rib parts for each rib;
generating a visualization of the ribs individually or as rib pars; and
generating an augmented visualization for out of plane rib parts.
